(19) United States
(12) Patent Application Publication
(10) Pub. No.: US 2011/0130679 A1
(43) Pub. Date: Jun. 2, 2011

(54) ALL-IN-ONE OPTICAL MICROSCOPIC HANDLE

(76) Inventors: Reuven Breslauer, Ashkelon (IL); Oded Nahlieli, Ashkelon (IL)

(21) Appl. No.: 12/377,803

(22) PCT Filed: Aug. 19, 2007
(86) PCT No.: PCT/IL07/01017

§ 371 Date: Feb. 17, 2009

(30) Foreign Application Priority Data
Aug. 17, 2006 (IL) 177550

(51) Int. Cl. A61B 10/02

(52) U.S. Cl. 600/566

(57) ABSTRACT

The present invention discloses an all-in-one fine needle aspiration means (FNA) for precision guidance and improved control of needle for ease of insertion, manipulation and efficient extraction of sample and a method thereof. Said FNA comprises means selected from a mechanically or electronically stabilized effecter; a mechanical or automated needle insertion and manipulation mechanism; optical means; a mechanical or automated vacuum or suction mechanism; and a biopsy collection receptacle.

### FIELD OF THE INVENTION

The present invention generally relates to an all-in-one optical microscopic handle, and more specifically, to fine needle aspiration devices, and to methods handling the same.

### BACKGROUND OF THE INVENTION

Fine needle aspiration (FNA) is a percutaneous (“through the skin”) procedure that uses a fine gauge needle (22 or 25 gauge) and a syringe to sample fluid from a breast cyst or remove clusters of cells from a solid mass. With FNA, the cellular material taken from the breast is usually sent to the pathology laboratory for analysis. The needle used during FNA is smaller than a needle that is normally used to draw blood. If the radiologist or surgeon just drains fluid from a cyst and does not send the sample to the pathology laboratory for analysis, the procedure is simply called cyst aspiration. Prior to FNA, the skin of the breast is cleansed and then may be anesthetized with a small hypodermic needle. If a breast lump can be felt, the radiologist or surgeon will guide a needle into the area of concern by palpating (feeling) the lump. If the lump is non-palpable (cannot be felt), the FNA procedure will be done under image-guidance using either stereotactic mammography or ultrasound with the patient in either the upright or prone (face down) position. Stereotactic mammography involves using computers to pinpoint the exact location of a breast mass based on mammograms (x-rays) taken from two different angles. The computer coordinates will help the physician to guide the needle to the correct area in the breast. With ultrasound, the radiologist or surgeon will watch the needle on the ultrasound monitor to help guide it to the area of concern. FNA is usually performed under ultrasound image guidance.

After the needle is placed into the breast in the region of the lesion (abnormality), a vacuum is created and multiple in and out needle motions are performed. Several needle insertions are usually required to ensure that an adequate tissue sample is taken. The samples are then smeared on a microscope slide and are: (i) allowed to dry in air, (ii) are “fixed” by spraying, or (iii) are immersed in a liquid. The fixed smears are then stained and examined by a pathologist under the microscope.

FNA does not require stitches and can usually be performed on an outpatient basis. A very small bandage is placed over the area after the procedure. Many patients resume their normal lifestyle and routine the same day of the FNA procedure. The effectiveness of FNA is largely operator-dependent; it requires a skilled radiologist or surgeon who has gained experience by performing several cases.

The art contains numerous devices and methods for conducting FNA. Focusing on a range of improvements from the mechanical stabilization of the needle to image guided insertion of the needle into suspected tissue. Methods for obtaining and examining ductal fluid from patients at cancer risk is described in U.S. Pat. Nos. 6,642,010, 6,638,727. The mechanical stabilization of the needle in FNA is addressed in U.S. Pat. Nos. 5,241,969, 4,594,073, US design D411882, WO patent 9325147 and Australia Patent 2658292.

Pencil-grip fine needle aspiration holder for stabilization and ease of manipulation is provided in U.S. Pat. 5,830,152. An improved disposable single-hand controlled aspirating device for medical aspirating activities is disclosed in U.S. Pat. 5,115,816. A finger ring assisted stabilization of FNA is described in U.S. Pat. 3,595,217. A handle of a FNA biopsy device, which is capable of preventing an ovary from being pierced by mistake in puncture when sucking the content liquid of an ovarian cyst is described in Japan patent application 2005110905. A motorized biopsy needle positioner employed in a mammography needle biopsy system is provided in U.S. Pat. Nos. 6,022,325, 5,735,264 and Taiwan patent 0497966. A mechanical guidance apparatus for use in FNA is provided in U.S. Pat. Nos. 6,447,477, 6,228,049 and 6,203,524. A device and method for precisely positioning a probe tip relative to a target lesion in a bodily protuberance by using a radiolucent platform is described in U.S. Pat. No. 5,056,523. Spring assisted needle insertion mechanism is described in European Patent EP0983021A2. A motorized biopsy needle positioner employed in a mammographic needle biopsy system receives coordinate information representative of an identified point of interest with offset compensation is provided in U.S. Pat. No. 5,803,912. A pneumatic insertion method and apparatus assisted by visual guidance is provided in WO patent 02051324 and US patent application 20020082518 and U.S. Pat. 6,527,731.

Apparatus and methods are provided for positioning a needle or cannula of a biopsy device for insertion into a tissue mass by correlating, in real-time, the actual needle or cannula position prior to insertion with its probable trajectory once inserted is described in U.S. Pat. Nos. 5,833,627 and 5,660,185. Vibration assisted needle insertion mechanism is described in U.S. Pat. No. 7,008,383. Mechanical and automation assisted needle insertion mechanisms are provided in WO patent 8907911. A heatable needle for increased ease of insertion is disclosed in WO patent 05096953 and US patent application 20050228312. An ingestible capsule based FNA device is suggested in WO patents 02100256A2, 02100256A3 as well as U.S. Pat. No. 7,008,383. High accuracy Magnetic resonance spectroscopy (MRS) assisted diagnosis of FNA samples is provided in New Zealand patent 0518529 and Canada patent 2388757. Methods and apparatus for cauterizing predetermined volumes of abnormal biological tissue using a user selectable or adjustable energy applicator is provided in U.S. Pat. Nos. 6,312,428, 6,106,524 and 5,947,964. Vacuum tube syringe biopsy apparatus is explained in U.S. Pat. Nos. 5,027,827, 5,651,372 and 5,246,011 and Canada patent 2432949. In situ electrical current measurement based characterization of suspected tissue is described in U.S. Pat. Nos. 5,928,159 and 5,630,426. A combined tissue extraction and analysis device is described in U.S. Pat. No. 4,697,600. An MRI assisted computer controlled endoscopic examination probe containing sensors and imaging technology is described in U.S. Pat. No. 6,871,086 and 5,732,150. Method and apparatus for radiopharmaceutical-guided biopsy is described in U.S. Pat. No. 5,961,457. A combined mammography and ultrasonic transducer imaging technology of the internal structure of breast tissue that are in geometric registration with a mammogram is described in U.S. Pat. Nos. 5,938,613, 5,479,927, 5,474,072 and 5,415,169. A device for aspiration of sample tissue is provided in WO patents 0211613 and 0211613. An aspiration needle for use in collecting large cell samples with a source of vacuum for fine needle aspiration cytology without increasing the size of the needle is described in WIFO patents 9632146, 9416626 and 06036108 as well as U.S. Pat. Nos. 5,902,279, 5,330,443, 5,199,441 and EU patents 0820317, 0695199 and Canada patent 2160707; and a kit thereof with syringe in WIFO patent 9423774 and U.S. Pat. Nos. 5,645,537 and 6,642,010. A method for collecting and concentrating cells in a fluid such as body fluid, liquid for irrigation or aspiration cytological material, wherein the fluid is brought to pass through a brush or a wad of cotton in the flow passage of the liquid for collecting existing cells on the filter body is found in WO patent 9200039. A device, to reciprocate and/or rotate at the same time causing tissue to enter the needle in which depth and number of the thrusts can be pre-programmed, and the force behind each thrust is constant is described in U.S. Pat. Nos. 6,689,072, 6,402,701, 5,060,658, 4,989,614 and European patent 1639947. A standardized automated collection method for easy cross referencing of samples is provided in WO patent 02100279. Cell traps for cell cluster, tissue and debris harvesting and collecting are used with an endoscope such that the fluid jets draw-in, stabilize and hold in-vivo target specimens at a distal end. While the catheter grips the tissue by suction, blasts of high pressure solution strip from the tissue and suspend clusters of cells which are ready for analysis is disclosed in U.S. Pat. No. 6,572,578. A safety syringe that poses less risk of accidental puncture on extraction is provided in U.S. Pat. No. 5,964,735. An upright patient position combined imaging and biopsy apparatus is provided in U.S. Pat. No. 5,386,447. A seated position combined imaging and biopsy apparatus is provided in U.S. Pat. No. 5,820,552. Traction-inducing compression assembly for enhanced tissue imaging is described in U.S. Pat. No. 5,851,180. A device for combining tomographic images with human vision using a half-silvered mirror to merge the visual outer surface of an object is provided U.S. Pat. No. 6,599,247. An ultrasound imaging and biopsy system is provided in which an upper compression member of the system includes a thin, flexible, sterile and disposable membrane that compresses the tissue and permits a biopsy instrument to be readily inserted therethrough is described in U.S. Pat. No. 6,027,457. Tissue extraction via an elongated cutting member is provided in U.S. Pat. Nos. 5,857,982 and 5,817,034. Hand gun shaped tissue extraction via a cutting member is disclosed in U.S. Pat. No. 6,036,657 and US design. A hand gun shaped FNA device with maneuverable and lockable needle position is provided in U.S. Pat. No. 5,469,860. An apparatus system and method for cauterizing a volume of tissue wherein electrode assemblies are deployed from the forward end region of an instrument in conjunction with an electrosurgical cutting activity is described in U.S. Pat. No. 6,287,304. A tissue tract sealant delivery device is provided in U.S. Pat. No. 6,840,952.

Much of the art addresses a single aspect of the FNA procedure (i.e. penetration, stabilization, sample collection, imaging direction, etc.). Moreover, art that does combine improvements tend to be cumbersome and difficult to handle.

### SUMMARY OF THE INVENTION

It would be desirable to achieve an all-in-one compact and easy to handle fine needle aspiration (NFA) device integrating manual and automated stabilization of needle, drug and antiseptic wash administration, improved needle penetration, improved sample collection by multi axel maneuverability of the inserted needle including rotation and assisted by vacuum or suction, needle safety features for the prevention of accidental punctures, improved mechanical and image guidance for precise insertion, integrated ultrasonic or endoscope camera probe with image enhancement and image stabilizer and finally, tract filler administrator.

Therefore, in accordance with a preferred embodiment of the present invention, there is provided a system adapted to control the operation of an FNA biopsy device comprising a mechanically or electronically stabilized needle, a mechanical or automated needle insertion and manipulation mechanism, a mechanical or automated vacuum or suction mechanism and a biopsy collection receptacle.

In a preferred embodiment of the invention there is further provided a drug and antiseptic administration duct.

In a further preferred embodiment of the invention there is further provided a contact endoscope or a probe that enables imaging of the suspect tissue for better precision both prior to penetration and in the collection of the sample.

In yet another preferred embodiment of the invention the duct created by the penetration of the needle can be sealed with a biocompatible sealant.

There is further provided a quick extraction enabled recto-anal shaft insert, additionally fitted with a peel-away mechanism that provides for the easy extraction of the insert.

In yet another preferred embodiment of the invention a cutting member replaces the needle.

The device of the present invention has many technology advantages, among them: Compactness; Ease of use; Integrated all-in-one procedure operation tool; Improved collection of sample; Improved mechanical and automated stabilization; and Improved imaging including incorporating image stabilization.

Additional features and advantages of the invention will become apparent from the following drawings and description.

It is in the scope of the present invention to provide an all-in-one fine needle aspiration means (FNA) for precision guidance and improved control of needle for ease of insertion, manipulation and efficient extraction of sample. The FNA comprises means selected inter alia from a mechanically or electronically stabilized effecter; a mechanical or automated needle insertion and manipulation mechanism; optical means; a mechanical or automated vacuum or suction mechanism; and a biopsy collection receptacle. The effecter is possibly at least one needle. The FNA may comprise needle safety features for the prevention of accidental punctures, improved mechanical and image guidance for precise insertion. The FNA may be adapted for improved sample collection comprising means for multi axel maneuverability of said inserted effecter. The effecter is adapted to rotate or other wise 3D maneuvers. The effecter is possibly assisted by vacuum or suction. The FNA may further comprise means for drug and antiseptic wash administration. The FNA may further comprise a contact endoscope or a probe adapted for imaging of suspect tissue. The FNA may further comprise a means for collecting of suspect tissue. The biopsy collection receptacle is possibly selected from a group including inter alia at least one vacuum extractor, biopsy forceps, bipolar cautery, resecting loop, stylet, electrode, flexible snare, electrocoagulating means, cutting member, blades, brushes, needles or the like or any combination thereof. The FNA may comprise means for sealing duct that was created by the penetration of the effecter, especially with biocompatible sealant. The FNA may comprise needle tract biocompatible filler administrator. The FNA may comprise means for a quick biopsy extraction, especially enabled recto-anal shafts insert. The FNA may comprise collecting means, especially a sample's container. The FNA may comprise an effecter adapted for medicaments injection, pigments of dyes introduction, tattoos application, indicators application or a combination thereof. The FNA may comprise a peel-away mechanism that provides for the easy extraction of the insert; at least one light source; optic guide; fiber optics; means for emitting laser beam; e.g., diode laser or any combination thereof. The FNA may comprise integrated ultrasonic or endoscope camera probe with image enhancement and image stabilization. The optical means may be selected from magnifying means, especially in power of 2 to 200. The optical means may be selected from magnifying means, especially in power of 200 to 1000. The optical means may include camera, especially CCD or other video camera.

Another object of the present invention is to provide in an all-in-one fine needle aspiration means (FNA) adapted for precision guidance and improved control of needle for ease of insertion, manipulation and efficient extraction of sample, an FNA comprising means for controlling a mechanically or electronically stabilized effecter; optical means; a mechanical or automated needle insertion and manipulation mechanism; a mechanical or automated vacuum or suction mechanism; and/or a biopsy collection receptacle. The effecter is possibly at least one needle. The FNA may comprise needle safety features for the prevention of accidental punctures, improved mechanical and image guidance for precise insertion. The FNA may be adapted for improved sample collection comprising means for multi axel maneuverability of said inserted effecter. The effecter is adapted to rotate or other wise 3D maneuvers. The effecter is possibly assisted by vacuum or suction. The FNA may further comprise means for drug and antiseptic wash administration. The FNA may further comprise a contact endoscope or a probe adapted for imaging of suspect tissue. The FNA may further comprise a means for collecting of suspect tissue. The biopsy collection receptacle is possibly selected from a group including inter alia at least one vacuum extractor, biopsy forceps, bipolar cautery, resecting loop, stylet, electrode, flexible snare, electrocoagulating means, cutting member, blades, brushes, needles or the like r any combination thereof. The FNA may comprise means for sealing duct that was created by the penetration of the effecter, especially with biocompatible sealant. The FNA may comprise needle tract biocompatible filler administrator. The FNA may comprise means for a quick biopsy extraction, especially enabled recto-anal shafts insert. The FNA may comprise collecting means, especially a sample's container. The FNA may comprise an effecter adapted for medicaments injection, pigments of dyes introduction, tattoos application, indicators application or a combination thereof. The FNA may comprise a peel-away mechanism that provides for the easy extraction of the insert; at least one light source; optic guide; fiber optics; means for emitting laser beam; e.g., diode laser or any combination thereof. The term ‘fiber optics’ refers also to a bundle (e.g., 50000 fibers for example) or an array of fibers provided in a manner that a high signal is provided. The FNA may comprise integrated ultrasonic or endoscope camera probe with image enhancement and image stabilization. The optical means may be selected from magnifying means, especially in power of 2 to 200. The optical means may be selected from magnifying means, especially in power of 200 to 1000. The optical means may include camera, especially CCD or other video camera.

Another object of the present invention is to disclose a method of providing an all-in-one fine needle aspiration means (FNA) for precision guidance and improved control of needle for ease of insertion, manipulation and efficient extraction of sample, comprising; stabilizing mechanically or electronically the effecter; inserting and manipulating the mechanical or automated needle mechanism; applying an image; operating the mechanical or automated vacuum or suction mechanism; collecting the biopsy receptacle.

The method may further comprise a step of assembling of the effecter with least one needle. The method may further comprise a step of assembling needle safety features for the prevention of accidental punctures. The method may further comprise a step of providing means for multi axel maneuverability of said inserted effecter for improved sample collection. The method may further comprise a step of adapting said effecter to rotating or other wise 3D maneuvering. The method may further comprise a step of assisting said effecter by vacuum or suction. The method may further comprise a step of administrating means for drug and antiseptic wash. The method may further comprise a step of adding a contact endoscope or a probe adapted for imaging of suspect tissue. The method may further comprise a step of adding means for collecting of suspect tissue. The method may further comprise a step of selecting the biopsy collection receptacle from a group including inter alia at least one vacuum extractor, biopsy forceps, bipolar cautery, resecting loop, stylet, electrode, flexible snare, electrocoagulating means, cutting member, needles or the like r any combination thereof. The method may further comprise a step of providing means for sealing duct created by the penetration of the effecter, especially with biocompatible sealant. The method may further comprise a step of providing needle tract biocompatible filler administrator. The method may further comprise a step of providing means for a quick biopsy extraction, especially enabled recto-anal shafts insert. The method may further comprise a step of providing a peel-away mechanism for the easy extraction of the insert. The method may further comprise a step of providing at least one light source. The method may further comprise a step of providing optic guide. The method may further comprise a step of providing fiber optics. The method may further comprise a step of providing means for emitting laser beam. The method may further comprise a step of providing diode laser as said means. The method may further comprise a step of providing integrated ultrasonic or endoscope camera probe with image enhancement and image stabilization. The method may further comprise a step of obtaining at least partially flexible FNA. The method may further comprise a step of magnifying the image especially in power of 2 to 200. The method may further comprise a step of magnifying the image especially in power of 200 to 1000. The method may further comprise a step of applying of an imade os provided by in-line or off-line utilizing wireless or cordial communicated CCD or other video camera. The method may further comprise a step of applying a collecting means, especially a sample's container. The method may further comprise a step of applying an effecter adapted for medicaments injection, pigments of dyes introduction, tattoos application, indicators application or a combination thereof.

Another object of the present invention is to disclose a method for utilizing a handset of an all-in-one fine needle aspiration means (FNA), comprising step or steps of providing adaptation for precision guidance and improved control of needle for ease of insertion, manipulation and efficient extraction of sample, an FNA, providing an image and additionally providing means for controlling a mechanically or electronically stabilized effecter; a mechanical or automated needle insertion and manipulation mechanism; a mechanical or automated vacuum or suction mechanism; and/or a biopsy collection receptacle.

The method may further comprise a step of assembling of the effecter with least one needle. The method may further comprise a step of assembling needle safety features for the prevention of accidental punctures. The method may further comprise a step of providing means for multi axel maneuverability of said inserted effecter for improved sample collection. The method may further comprise a step of adapting said effecter to rotating or other wise 3D maneuvering. The method may further comprise a step of assisting said effecter by vacuum or suction. The method may further comprise a step of administrating means for drug and antiseptic wash. The method may further comprise a step of adding a contact endoscope or a probe adapted for imaging of suspect tissue. The method may further comprise a step of adding means for collecting of suspect tissue. The method may further comprise a step of selecting the biopsy collection receptacle from a group including inter alia at least one vacuum extractor, biopsy forceps, bipolar cautery, resecting loop, stylet, electrode, flexible snare, electrocoagulating means, cutting member, needles or the like r any combination thereof. The method may further comprise a step of providing means for sealing duct created by the penetration of the effecter, especially with biocompatible sealant. The method may further comprise a step of providing needle tract biocompatible filler administrator. The method may further comprise a step of providing means for a quick biopsy extraction, especially enabled recto-anal shafts insert. The method may further comprise a step of providing a peel-away mechanism for the easy extraction of the insert. The method may further comprise a step of providing at least one light source. The method may further comprise a step of providing optic guide. The method may further comprise a step of providing fiber optics. The method may further comprise a step of providing means for emitting laser beam. The method may further comprise a step of providing diode laser as said means. The method may further comprise a step of providing integrated ultrasonic or endoscope camera probe with image enhancement and image stabilization. The method may further comprise a step of obtaining at least partially flexible FNA. The method may further comprise a step of magnifying the image especially in power of 2 to 200. The method may further comprise a step of magnifying the image especially in power of 200 to 1000. The method may further comprise a step of applying of an imade os provided by in-line or off-line utilizing wireless or cordial communicated CCD or other video camera. The method may further comprise a step of applying a collecting means, especially a sample's container. The method may further comprise a step of applying an effecter adapted for medicaments injection, pigments of dyes introduction, tattoos application, indicators application or a combination thereof.

The FNA and/or FNA handset is possibly flexible and/or disposable. The handset may have an ergonomic hand gun shaped enclosure or a pen shaped enclosure with a pen grip incorporated.

### BRIEF DESCRIPTION OF THE DRAWINGS

For a better understanding of the invention with regard to the embodiments thereof, reference is made to the accompanying drawings (not to scale), in which like numerals designate corresponding sections or elements throughout, and in which:

FIG. 1 is a general view an all-in-one FNA sample collection apparatus including imaging technology, drug and antiseptic administration capacity and suction assisted needle based tissue sampler;

FIG. 2 is another general view of a general view an all-in-one FNA sample collection apparatus including imaging technology, drug and antiseptic administration capacity and suction assisted needle based tissue sampler; and,

FIG. 3 is another general view of a general view an all-in-one FNA sample collection apparatus including a sample container.

### DETAILED DESCRIPTION OF THE PREFERRED EMBODIMENTS

The all-in-one tissue sampling device is useful for the extraction of liquid samples and their subsequent analysis. More specifically it may be utilized in medical procedures that require the acquirement of suspect tissue for the purpose of testing its characteristics. Combing advanced stabilized imaging technology with mechanical and automated precision penetration devices, multi directional maneuverability of the sharpened member or needle, vacuum or suction assisted extraction of the tissue, as well as the ability to administer drugs and antisepsis to the area and a filler to the invasive duct formed by the member, it improves the ratio of success in this highly user dependent procedure.

The present invention discloses an all-in-one fine needle aspiration means (FNA) for precision guidance and improved control of needle for ease of insertion, manipulation and efficient extraction of sample. The FNA is preferably comprised of means selected from (i) a mechanically or electronically stabilized effecter; (ii) a mechanical or automated needle insertion and manipulation mechanism; (iii) a mechanical or automated vacuum or suction mechanism; and (iv) a biopsy collection receptacle.

It is in the scope of the present invention wherein the effecter is at least one needle. The FNA may contain needle safety features for the prevention of accidental punctures, improved mechanical and image guidance for precise insertion. It may be adapted for improved sample collection comprising means for multi axel maneuverability of said inserted effecter. The effecter is possibly adapted to rotate or otherwise 3D maneuvers. The effecter is possibly assisted by vacuum or suction.

It is also in the scope of the present invention wherein the FNA further comprises means for drug and antiseptic wash administration. It may further comprise a contact endoscope or a probe adapted for imaging of suspect tissue. It may further comprise a means for collecting of suspect tissue.

The biopsy collection receptacle is selected in a non-limiting manner from a group including inter alia at least one vacuum extractor, biopsy forceps, bipolar cautery, resecting loop, stylet, electrode, flexible snare, electrocoagulating means, cutting member, needles or the like or any combination thereof. The FNA may additionally comprise means for sealing duct that was created by the penetration of the effecter, especially with biocompatible sealant. It may possibly comprise a needle tract biocompatible filler administrator.

The FNA may further comprise means for a quick biopsy extraction, especially enabled recto-anal shafts insert. It may further comprise a peel-away mechanism that provides for the easy extraction of the insert. It may further comprise at least one light source. It may further comprise optic guide. It may further comprise fiber optics. It may further comprise means for emitting laser beam. This means is preferably a diode laser.

It is also in the scope of the present invention wherein the FNA further compris integrated ultrasonic or endoscope camera probe with image enhancement and image stabilization. At least portion of the aforesaid FNA is made or flexible materials so as the FNA is flexible.

It is another object of the present invention to disclose an all-in-one fine needle aspiration means (FNA) adapted for precision guidance and improved control of needle for ease of insertion, manipulation and efficient extraction of sample, an FNA comprising means for controlling a mechanically or electronically stabilized effecter; a mechanical or automated needle insertion and manipulation mechanism; a mechanical or automated vacuum or suction mechanism; and/or a biopsy collection receptacle. The FNA handset is preferably provided wherein the effecter is at least one needle. It is possibly further adapted for controlling a means for drug and antiseptic administration. It is possibly further adapted for controlling means for collecting of suspect tissue. It is possibly further adapted for controlling a means for biopsy collection receptacle, wherein the collection receptacle is selected in a non-limiting manner from a group including inter alia at least one vacuum extractor, biopsy forceps, bipolar cautery, resecting loop, stylet, electrode, flexible snare, electrocoagulating means, cutting member, needles or the like or any combination thereof.

The FNA handset may further be adapted for controlling means for sealing duct that was created by the penetration of the effecter, especially with biocompatible sealant. It may be further adapted for controlling means for a quick biopsy extraction, especially enabled recto-anal shafts insert. It may be further adapted for controlling means adapted for a peel-away mechanism that provides for the easy extraction of the insert. It may control at least one light source. It may be adapted for controlling an optic guide. It may additionally comprise one or more fiber optics. It may be further adapted for controlling means for emitting laser beam, especially a diode laser. It may be comprising needle safety features for the prevention of accidental punctures, improved mechanical and image guidance for precise insertion. It may be further adapted for improved sample collection comprising means for multi axel maneuverability of said inserted effecter. It may be adapted to rotate or otherwise 3D maneuvers this effecter. It may additionally comprise means for controlling integrated ultrasonic or endoscope camera probe with image enhancement and image stabilization. This FNA handset is possibly a flexible member. It may be further incorporated in an ergonomic hand gun shaped enclosure. It may be further be characterized by a pen shaped enclosure comprising a pen grip incorporated. The aforesaid needle penetration is heat assisted. It may be further be adapted so as it is being conveyed with assistance of a computer controlled robotic arm.

Another object of the present invention is to disclose a method of providing an all-in-one fine needle aspiration means (FNA) for precision guidance and improved control of needle for ease of insertion, manipulation and efficient extraction of sample, comprising; stabilizing mechanically or electronically the effecter; inserting and manipulating the mechanical or automated needle mechanism; operating the mechanical or automated vacuum or suction mechanism; collecting the biopsy receptacle. It is in the scope of the present invention wherein the FNA is as defined in any of the above.

Another object of the present invention is to disclose a method for utilizing an all-in-one fine needle aspiration means (FNA), comprising step or steps of providing adaptation for precision guidance and improved control of needle for ease of insertion, manipulation and efficient extraction of sample, an FNA, additionally providing means for controlling a mechanically or electronically stabilized effecter; a mechanical or automated needle insertion and manipulation mechanism; a mechanical or automated vacuum or suction mechanism; and/or a biopsy collection receptacle. It is in the scope of the present invention wherein the FNA is as defined in any of the above.

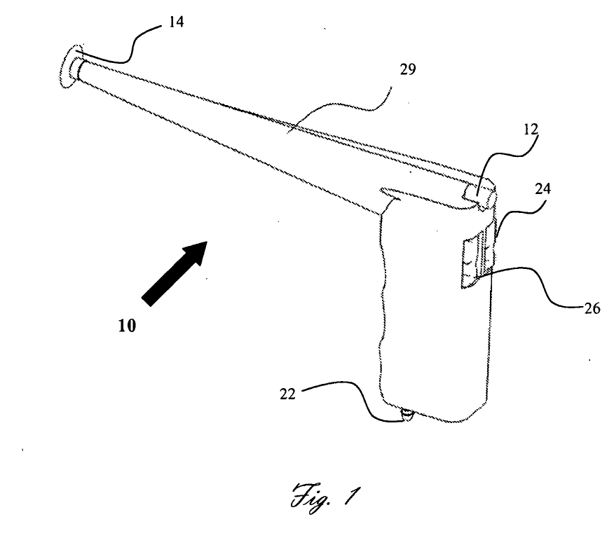

FIG. 1 is a general view of an all-in-one FNA sample collection apparatus 10 comprising shaft 29 containing extendable needle (not shown) to be extended through adhesion cap 14. Insertion is visually assisted by contact endoscope 12 that runs through shaft 29 reaching adhesion cap 14. Prior to insertion of the needle (not shown) the user may administer drugs and antiseptics to the suspect area by placing the drugs in drug compartment 26 that are channeled through duct (not shown) running through shaft 29 to adhesion cap 14, or oxygen supplied via input jack 22 and conveyed through ducting (not shown) to adhesion cap 14. Once user has been visually guided by means of contact endoscope 12 to the precise area the needle (not shown) can be protruded from adhesion cap 14 and further manipulated by mechanical or automated mechanism (not shown) to achieve the required sample collection in biopsy receptacle 24.

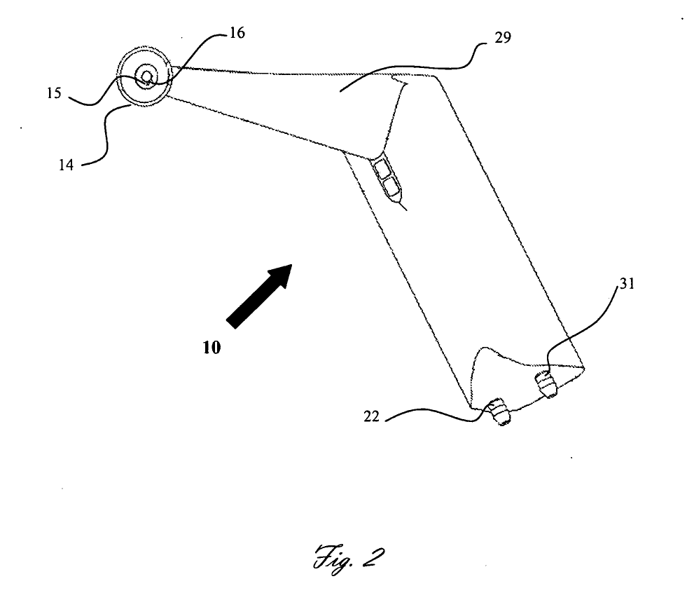

FIG. 2 is yet another general view of an all-in-one FNA sample collection apparatus 10 comprising shaft 29 containing extendable needle (not shown) to be extended through bore 15 in adhesion cap 14. Insertion is visually assisted by contact endoscope 12 that runs through shaft 29 reaching adhesion cap 14. Prior to insertion of the needle (not shown) the user may administer drugs and antiseptics to the suspect area by placing the drugs in drug compartment (not shown) that are channeled through duct (not shown) running through shaft 29 to bore 16 in adhesion cap 14. Once user has been visually guided by means of contact endoscope 12 to the precise area the needle (not shown) can be protruded from bore 15 in adhesion cap 14 and further manipulated by mechanical or automated mechanism (not shown) to achieve the required sample collection. Further suction can be administered by means of a suction device connected to.

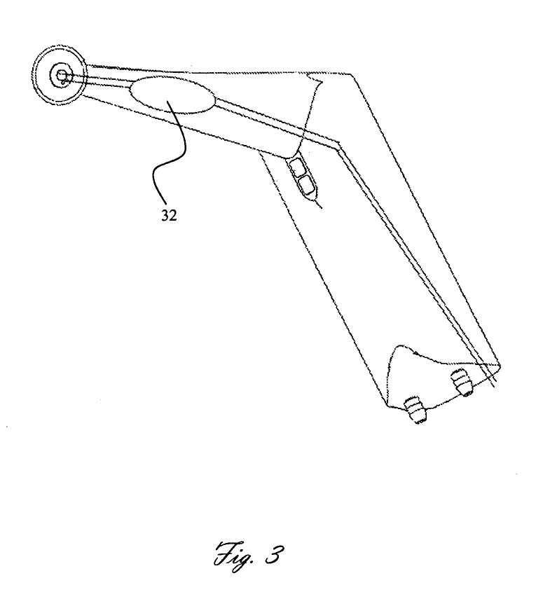

FIG. 3 is yet another general view of a general view an all-in-one FNA sample collection apparatus including a sample container 32.

It is in the scope of the invention wherein an all-in-one fine needle aspiration means (FNA) for precision guidance and improved control of needle for ease of insertion, manipulation and efficient extraction of sample is provided. The FNA comprising means selected from:
a. a mechanically or electronically stabilized effecter;
b. a mechanical or automated needle insertion and manipulation mechanism;
c. optical means;
d. a mechanical or automated vacuum or suction mechanism; and
e. a biopsy collection receptacle.

It is also in the scope of the invention wherein the effecter is at least one needle, and/or wherein the FNA comprising needle safety features for the prevention of accidental punctures, improved mechanical and image guidance for precise insertion.

It is also in the scope of the invention wherein the FNA is adapted for improved sample collection comprising means for multi axel maneuverability of said inserted effecter.

It is also in the scope of the invention wherein the effecter is adapted to rotate or other wise 3D maneuver.

It is also in the scope of the invention wherein the effecter is assisted by vacuum or suction.

It is also in the scope of the invention wherein the FNA further comprising means for drug and antiseptic wash administration.

It is also in the scope of the invention wherein the FNA further comprising a contact endoscope or a probe adapted for imaging of suspect tissue.

It is also in the scope of the invention wherein the FNA further comprising a means for collecting of suspect tissue.

It is also in the scope of the invention wherein the said biopsy collection receptacle is selected from a group including inter alia at least one vacuum extractor, biopsy forceps, bipolar cautery, resecting loop, stylet, electrode, flexible snare, electrocoagulating means, cutting member, blades, brushes, needles or the like r any combination thereof.

It is also in the scope of the invention wherein the FNA additionally comprising means for sealing duct that was created by the penetration of the effecter, especially with biocompatible sealant.

It is also in the scope of the invention wherein the FNA comprising needle tract biocompatible filler administrator.

It is also in the scope of the invention wherein the FNA additionally comprising means for a quick biopsy extraction, especially enabled recto-anal shafts insert.

It is also in the scope of the invention wherein the FNA additionally comprising collecting means, especially a sample's container.

It is also in the scope of the invention wherein the FNA additionally comprising an effecter adapted for medicaments injection, pigments of dyes introduction, tattoos application, indicators application or a combination thereof.

It is also in the scope of the invention wherein the FNA additionally comprising a peel-away mechanism that provides for the easy extraction of the insert.

It is also in the scope of the invention wherein the FNA additionally comprising at least one light source.

It is also in the scope of the invention wherein the FNA additionally comprising optic guide.

It is also in the scope of the invention wherein the FNA additionally comprising fiber optics, bundle of optic fibers or an array thereof.

It is also in the scope of the invention wherein the FNA additionally comprising means for emitting laser beam, e.g., a diode laser.

It is also in the scope of the invention wherein the FNA additionally comprising integrated ultrasonic or endoscope camera probe with image enhancement and image stabilization.

It is also in the scope of the invention wherein the said optical means is selected from magnifying means, especially in power of 2 to 200.

It is also in the scope of the invention wherein the optical means is selected from magnifying means, especially in power of 200 to 1000.

It is also in the scope of the invention wherein the said optical means including camera, especially CCD or other video camera.

It is also in the scope of the invention wherein the FNA handset which is adapted for precision guidance and improved control of needle for ease of insertion, manipulation and efficient extraction of sample, an FNA comprising means for controlling a mechanically or electronically stabilized effecter; optical means; a mechanical or automated needle insertion and manipulation mechanism; a mechanical or automated vacuum or suction mechanism; and/or a biopsy collection receptacle.

It is also in the scope of the invention wherein this FNA handset is adapted in a manner that the effecter is at least one needle.

It is also in the scope of the invention wherein this FNA handset is adapted for controlling means for drug and antiseptic administration.

It is also in the scope of the invention wherein this FNA handset is adapted for controlling means for collecting of suspect tissue.

It is also in the scope of the invention wherein this FNA handset is adapted for controlling means for biopsy collection receptacle, wherein said collection receptacle is selected from a group including inter alia at least one vacuum extractor, biopsy forceps, bipolar cautery, resecting loop, stylet, electrode, flexible snare, electrocoagulating means, cutting member, needles or the like or any combination thereof.

It is also in the scope of the invention wherein this FNA handset is adapted controlling means for sealing duct that was created by the penetration of the effecter, especially with biocompatible sealant.

It is also in the scope of the invention wherein this FNA handset is adapted for controlling means for a quick biopsy extraction, especially enabled recto-anal shafts insert.

It is also in the scope of the invention wherein this FNA handset is adapted for controlling means adapted for a peel-away mechanism that provides for the easy extraction of the insert.

It is also in the scope of the invention wherein this FNA handset is adapted for controlling at least one light source.

It is also in the scope of the invention wherein this FNA handset is adapted for controlling an optic guide.

It is also in the scope of the invention wherein this FNA handset handset additionally comprising fiber optics, bundle of optic fibers or an array thereof.

It is also in the scope of the invention wherein this FNA handset is adapted for controlling means for emitting laser beam, especially a diode laser.

It is also in the scope of the invention wherein this FNA handset comprising needle safety features for the prevention of accidental punctures, improved mechanical and image guidance for precise insertion.

It is also in the scope of the invention wherein this FNA handset is adapted for improved sample collection comprising means for multi axel maneuverability of said inserted effecter.

It is also in the scope of the invention wherein this FNA handset is adapted rotating or otherwise 3D maneuvering.

It is also in the scope of the invention wherein this FNA handset comprising means for controlling integrated ultrasonic or endoscope camera probe with image enhancement and image stabilization.

It is also in the scope of the invention wherein this FNA handset is incorporated in an ergonomic hand gun shaped enclosure.

It is also in the scope of the invention wherein this FNA handset is characterized by a pen shaped enclosure comprising a pen grip incorporated.

It is also in the scope of the invention wherein this FNA handset comprising a needle penetration which is heat assisted.

It is also in the scope of the invention wherein this FNA handset being conveyed with assistance of a computer controlled robotic arm.

It is also in the scope of the invention wherein this FNA handset comprising optical means is selected from magnifying means, especially in power of 2 to 200.

It is also in the scope of the invention wherein this FNA handset is comprised of optical means which is selected from magnifying means, especially in power of 200 to 1000.

It is also in the scope of the invention wherein this FNA handset comprises an optical means that is including camera, especially CCD or other video camera.

It is also in the scope of the invention wherein this FNA handset is adapted additionally comprising collecting means, especially a sample's container.

It is also in the scope of the invention wherein this FNA handset additionally comprising an effecter adapted for medicaments injection, pigments of dyes introduction, tattoos application, indicators application or a combination thereof.

It is also in the scope of the invention to disclose a method of providing an all-in-one fine needle aspiration means (FNA) for precision guidance and improved control of needle for ease of insertion, manipulation and efficient extraction of sample, comprising; stabilizing mechanically or electronically the effecter; inserting and manipulating the mechanical or automated needle mechanism; applying an image; operating the mechanical or automated vacuum or suction mechanism; collecting the biopsy receptacle.

It is also in the scope of the invention wherein this method comprising a step of assembling of the effecter with least one needle.

It is also in the scope of the invention wherein this method comprising a step of assembling needle safety features for the prevention of accidental punctures.

It is also in the scope of the invention wherein this method comprising a step of providing means for multi axel maneuverability of said inserted effecter for improved sample collection.

It is also in the scope of the invention wherein this method comprising a step of adapting said effecter to rotating or other wise 3D maneuvering.

It is also in the scope of the invention wherein this method comprising a step of assisting said effecter by vacuum or suction.

It is also in the scope of the invention wherein this method comprising a step of administrating means for drug and antiseptic wash.

It is also in the scope of the invention wherein this method comprising a step of adding a contact endoscope or a probe adapted for imaging of suspect tissue.

It is also in the scope of the invention wherein this method comprising a step of adding means for collecting of suspect tissue.

It is also in the scope of the invention wherein this method comprising a step of selecting said biopsy collection receptacle from a group including inter alia at least one vacuum extractor, biopsy forceps, bipolar cautery, resecting loop, stylet, electrode, flexible snare, electrocoagulating means, cutting member, needles or the like r any combination thereof.

It is also in the scope of the invention wherein this method comprising a step of providing means for sealing duct created by the penetration of the effecter, especially with biocompatible sealant.

It is also in the scope of the invention wherein this method comprising a step of providing needle tract biocompatible filler administrator.

It is also in the scope of the invention wherein this method comprising a step of providing means for a quick biopsy extraction, especially enabled recto-anal shafts insert.

It is also in the scope of the invention wherein this method comprising a step of providing a peel-away mechanism for the easy extraction of the insert.

It is also in the scope of the invention wherein this method comprising a step of providing at least one light source.

It is also in the scope of the invention wherein this method comprising a step of providing optic guide.

It is also in the scope of the invention wherein this method comprising a step of providing fiber optics.

It is also in the scope of the invention wherein this method comprising a step of providing means for emitting laser beam.

It is also in the scope of the invention wherein this method comprising a step of providing diode laser as said means.

It is also in the scope of the invention wherein this method comprising a step of providing an integrated ultrasonic or endoscope camera probe with image enhancement and image stabilization.

It is also in the scope of the invention wherein this method comprising a step of obtaining at least partially flexible FNA.

It is also in the scope of the invention wherein this method comprising a step of magnifying the image especially in power of 2 to 200.

It is also in the scope of the invention wherein this method comprising a step of magnifying the image especially in power of 200 to 1000.

It is also in the scope of the invention wherein this method comprising a step of applying of an image provided by in-line or off-line utilizing wireless or cordial communicated CCD or other video camera.

It is also in the scope of the invention wherein this method comprising a step of applying a collecting means, especially a sample's container.

It is also in the scope of the invention wherein this method comprising a step of applying an effecter adapted for medicaments injection, pigments of dyes introduction, tattoos application, indicators application or a combination thereof.

A method for utilizing a handset of an all-in-one fine needle aspiration means (FNA), comprising step or steps of providing adaptation for precision guidance and improved control of needle for ease of insertion, manipulation and efficient extraction of sample, an FNA, providing an image and additionally providing means for controlling a mechanically or electronically stabilized effecter; a mechanical or automated needle insertion and manipulation mechanism; a mechanical or automated vacuum or suction mechanism; and/or a biopsy collection receptacle.

It is also in the scope of the invention wherein this method of utilizing FNA handset additionally comprising a step of providing the effecter with at least one needle.

It is also in the scope of the invention wherein this method of utilizing FNA handset additionally comprising a step of obtaining at least one controlling means for drug and antiseptic administration.

It is also in the scope of the invention wherein this method of utilizing FNA handset additionally comprising a step of obtaining at least one controlling means for collecting of suspect tissue.

It is also in the scope of the invention wherein this method of utilizing FNA handset additionally comprising a step of obtaining at least one controlling means for biopsy collection receptacle, wherein selecting said collection receptacle from a group including inter alia at least one vacuum extractor, biopsy forceps, bipolar cautery, resecting loop, stylet, electrode, flexible snare, electrocoagulating means, cutting member, needles or the like or any combination thereof.

It is also in the scope of the invention wherein this method of utilizing FNA handset additionally comprising a step of obtaining at least one controlling means for sealing duct created by the penetration of the effecter, especially with biocompatible sealant.

It is also in the scope of the invention wherein this method of utilizing FNA handset additionally comprising a step of obtaining at least one controlling means for a quick biopsy extraction, especially enabled recto-anal shafts insert.

It is also in the scope of the invention wherein this method of utilizing FNA handset additionally comprising a step of obtaining at least one controlling means for a peel-away mechanism that provides for the easy extraction of the insert.

It is also in the scope of the invention wherein this method of utilizing FNA handset additionally comprising a step of obtaining at least one controlling means for controlling at least one light source.

It is also in the scope of the invention wherein this method of utilizing FNA handset additionally comprising a step of obtaining at least one controlling means for controlling an optic guide.

It is also in the scope of the invention wherein this method of utilizing FNA handset additionally comprising a step of providing fiber optics.

It is also in the scope of the invention wherein this method of utilizing FNA handset additionally comprising a step of obtaining at least one controlling means for emitting laser beam, especially a diode laser.

It is also in the scope of the invention wherein this method of utilizing FNA handset additionally comprising a step of providing needle safety features for the prevention of accidental punctures and improved mechanical and image guidance for precise insertion.

It is also in the scope of the invention wherein this method of utilizing FNA handset additionally comprising a step of providing means for improved sample collection and means for multi axel maneuverability of said inserted effecter.

It is also in the scope of the invention wherein this method of utilizing FNA handset additionally comprising a step of maneuvering effecter to rotate or otherwise 3D maneuver.

It is also in the scope of the invention wherein this method of utilizing FNA handset additionally comprising a step of providing means for controlling integrated ultrasonic or endoscope camera probe with image enhancement and image stabilization.

It is also in the scope of the invention wherein this method of utilizing FNA handset additionally comprising a step of magnifying the image especially in power of 2 to 200.

It is also in the scope of the invention wherein this method of utilizing FNA handset additionally comprising a step of magnifying the image especially in power of 200 to 1000.

It is also in the scope of the invention wherein this method of utilizing FNA handset additionally comprising a step of obtaining an image provided by in-line or off-line utilizing wireless or cordial communicated CCD or other video camera.

It is also in the scope of the invention wherein this method of utilizing FNA handset additionally comprising a step of of applying a collecting means, especially a sample's container.

It is also in the scope of the invention wherein this method of utilizing FNA handset additionally comprising a step of applying an effecter adapted for medicaments injection, pigments of dyes introduction, tattoos application, indicators application or a combination thereof.

It is also in the scope of the invention wherein this method of utilizing FNA handset additionally comprising a step of obtaining at least partially flexible FNA handset.

It is also in the scope of the invention wherein this method of utilizing FNA handset additionally comprising a step of incorporating said FNA in an ergonomic hand gun shaped enclosure.

It is also in the scope of the invention wherein this method of utilizing FNA handset additionally comprising a step of incorporating said FNA in a pen shaped enclosure with a pen grip incorporated.

It is also in the scope of the invention wherein this method of utilizing FNA handset additionally comprising a step of assisting needle penetration by heat.

It is also in the scope of the invention wherein this method of utilizing FNA handset additionally comprising a step of conveying with the assistance of a computer controlled robotic arm.

1. An all-in-one fine needle aspiration means (FNA) for precision guidance and improved control of needle for ease of insertion, manipulation and efficient extraction of sample is provided, comprising:
a. a mechanically or electronically stabilized effecter;
b. a mechanical or automated needle insertion and manipulation mechanism;
c. optical means;
d. a mechanical or automated vacuum or suction mechanism; and,
e. a biopsy collection receptacle.

2. The FNA according to claim 1, wherein the FDA is adapted for improved sample collection comprising means for multi axel maneuverability of said inserted effecter.

3. The FNA according to claim 1, wherein the effecter is adapted to rotate or other wise 3D maneuver.

4. The FNA according to claim 1, wherein the effecter is assisted by vacuum or suction.

5. The FNA according to claim 1, further comprising means for drug and antiseptic wash administration.

6. The FNA according to claim 1, further comprising a contact endoscope or a probe adapted for imaging of suspect tissue.

7. The FNA according to claim 1, comprising a means for collecting of suspect tissue.

8. The FNA according to claim 1, wherein the said biopsy collection receptacle is selected from a group including inter alia at least one vacuum extractor, biopsy forceps, bipolar cautery, resecting loop, stylet, electrode, flexible snare, electrocoagulating means, cutting member, blades, brushes, needles or the like r any combination thereof.

9. The FNA according to claim 1, additionally comprising means for sealing duct that was created by the penetration of the effecter, especially with biocompatible sealant.

10. The FNA according to claim 1, comprising needle tract biocompatible filler administrator.

11. The FNA according to claim 1, additionally comprising means for a quick biopsy extraction, especially enabled recto-anal shafts insert.

12. The FNA according to claim 1, additionally comprising collecting means, especially a sample's container.

13. The FNA according to claim 1, additionally comprising an effecter adapted for medicaments injection, pigments of dyes introduction, tattoos application, indicators application or any combination thereof.

14. The FNA according to claim 1, additionally comprising a peel-away mechanism that provides for the easy extraction of the insert.

15. The FNA according to claim 1, additionally comprising at least one light source.

16. The FNA according to claim 1, additionally comprising optic guide.

17. The FNA according to claim 1, additionally comprising fiber optics, bundle of optic fibers or an array thereof.

18. The FNA according to claim 1, additionally comprising means for emitting laser beam, and especially a diode laser.

19. The FNA according to claim 1, additionally comprising integrated ultrasonic or endoscope camera probe with image enhancement and image stabilization.

20. The FNA according to claim 1, wherein the said optical means is selected from magnifying means, especially in power of 2 to 200.

21. The FNA according to claim 1, wherein the optical means is selected from magnifying means, especially in power of 200 to 1000.

22. The FNA according to claim 1, wherein the said optical means including camera, especially CCD or other video camera.

23. The FNA handset according to claim 1, adapted for precision guidance and improved control of needle for ease of insertion, manipulation and efficient extraction of sample, an FNA comprising means for controlling a mechanically or electronically stabilized effecter; optical means; a mechanical or automated needle insertion and manipulation mechanism; a mechanical or automated vacuum or suction mechanism; and/or a biopsy collection receptacle.

24. A method of providing an all-in-one fine needle aspiration means (FNA) for precision guidance and improved control of needle for ease of insertion, manipulation and efficient extraction of sample, comprising; stabilizing mechanically or electronically the effecter; inserting and manipulating the mechanical or automated needle mechanism; applying an image; operating the mechanical or automated vacuum or suction mechanism; collecting the biopsy receptacle.

25. A method for utilizing a handset of an all-in-one fine needle aspiration means (FNA), comprising step or steps of providing adaptation for precision guidance and improved control of needle for ease of insertion, manipulation and efficient extraction of sample, an FNA, providing an image and additionally providing means for controlling a mechanically or, electronically stabilized effecter; a mechanical or automated needle insertion and manipulation mechanism; a mechanical or automated vacuum or suction mechanism; and/or a biopsy collection receptacle.
